(19) United States
(12) Patent Application Publication
(10) Pub. No.: US 2017/0260554 A1
(43) Pub. Date: Sep. 14, 2017

(54) Method For The Enzyme-Catalyzed Production Of Prepolymers For Producing Plastics

(71) Applicants: Karlsruher Institut für Technologie, Eggenstein-Leopoldshafen (DE); BioPro Baden-Württemberg GmbH, Stuttgart (DE)

(72) Inventors: Christoph Syldatk, Karlsruhe (DE); Ralf Kindervater, Gechingen (DE)

(21) Appl. No.: 15/329,810

(22) PCT Filed: Jul. 29, 2015
(86) PCT No.: PCT/EP2015/067358

§ 371 Date: Jan. 27, 2017

(30) Foreign Application Priority Data
Jul. 31, 2014 (DE) 102014215081.7

(51) Int. Cl. C12P 13/02; C08G 69/08; C08G 63/06; C08G 63/82; C08G 63/16; C08G 69/28; C08G 69/04

(52) U.S. Cl. CPC C12P 13/02; C08G 69/28; C08G 69/08; C08G 69/04; C08G 63/82; C08G 63/16; C08G 63/06

(57) ABSTRACT

A process for the enzyme-catalyzed preparation of prepolymers for the production of plastics, based on an enzyme-catalyzed polymerization of monomer or oligomer compounds in a single phase aqueous solution, as well as the separation of the prepolymers precipitated therefrom and their subsequent use for the production of plastics and plastic articles obtainable therefrom. In particular, the invention relates to respective methods for enzyme-catalyzed preparation of prepolymers with polyamide-type bonding structure for the production of polyamide-based plastics.

### INTRODUCTION

The present invention relates to a process for the enzyme-catalyzed preparation of prepolymers for the production of plastics, based on an enzyme-catalyzed polymerization of monomer or oligomer compounds, as well as the prepolymers obtainable therefrom and their use for the production of plastics and plastic products obtainable therefrom. In particular, the invention relates to respective methods for enzyme-catalyzed production of prepolymers with polyamide bonding structure for the production of polyamide-based plastics.

### BACKGROUND

The current industrial main production process for plastic and plastic products is based almost exclusively on conventional petrochemical processes, wherein in large integrated production facilities huge amounts of chemical intermediates are generated using fossil fuels, which are then processed into monomers, raw polymers, fine polymers and the corresponding precursors of plastics processing, such as granules, films and semi-finished products, to be finally molded in the plastics industry to finished products or components.

Especially in view of the increasing shortage of resources and the associated increasing challenges to increasingly take into account the environmental and climate protection also in the field of industrial production of consumer goods, especially in the plastics industry, there is growing interest and need to develop and establish new ways of production with improved sustainability. In particular in view of the increasing shortage of resources a considerable interest exists to develop alternative methods that make it possible to reduce or even avoid completely the use of fossil resources. This kind of a new, soft chemistry requires to be able to use precursors from aqueous solutions without having to make high demands on the purity of the target substances.

For the aforementioned reasons, the interest and the need for alternative methods increases, wherein in these large scale chemical processes the use of petrochemical raw materials can be reduced or wherein petrochemical raw materials can be replaced by more sustainable raw materials, and thus be able to provide with increasing shortage of resources alternative resources and energy-efficient production processes, and thus in the long term be able to ensure the protection of industrial production routes.

Therein, important aspects constitute a process management being improved under ecological viewpoints as well as providing new methods for producing sustainable plastics, and in particular the production of bio-based plastics based on renewable raw materials.

### STATE OF THE ART

In principle, polymerization methods such as polycondensation for producing polymers for the plastics industry are known and in the conventional petrochemical methods of chemical polymerization usually applied in large scale industry, the process is carried out in organic solvents or in molten salts or by using elaborate anhydrous reactor systems or by using azeotropic distillation.

A disadvantage of these methods is on the one hand the under ecological viewpoints elaborate technical process management in complex reactor systems and on the other hand the need for the high purity of the precursors as well as the need of separating the organic non-polar solvent systems and the related need for disposal or recycling thereof.

The inventors of the present invention have now found a novel polymerization process for the preparation of prepolymers, which are suitable for the production of plastics, wherein a polymerization of suitable monomers and/or oligomers, being present in an aqueous solution, is carried out by enzyme-catalyzed polymerization to form the corresponding prepolymers, which are precipitated from the aqueous reaction solution.

The process of the invention is particularly well suitable for the enzyme-catalyzed preparation of prepolymers from e.g. bioengineered monomers or oligomers in order to produce bio-based plastics therefrom, which can be prepared by conventional petrochemical synthesis routes only via many process steps, thus not being economically reasonable.

In principle, methods for the production of bioplastics based on fully or partially bio-based polymers, wherein the fossil raw materials of established processes are increasingly replaced by renewable raw materials, are already known. Examples include bio-based polyethylene (Bio-PE), polypropylene (Bio-PP), polyester, and other bio-based polymers. Therein, also biotechnologically, e.g. by fermentation, prepared polymers (prepolymers), such as in particular polyesters prepared by fermentation, are already produced and used in the production of bioplastics. An example of a recombinantly prepared diaminopentane is known from WO 2009/092793, wherein diaminopentane (DAP) prepared by fermentation is isolated from a DAP-containing fermentation broth by alkalizing and thermally treating the fermentation broth followed by extraction of the DAP using an organic solvent and finally isolating it from the separated organic phase.

WO 2013/044076 A1 describes the fermentative production of acrylic acid and other carboxylic acid compounds.

The use of biotechnological processes for the production of polymers is of particular interest with regard to the precedural economy and the access to plastics with new product features which are so far difficult to obtain with petrochemical processes.

Generally, the principle of enzyme-catalyzed polymerization or enzymatic synthesis of oligomers is already known. For example, in the dissertation by M. Andre (“Chemoenzymatische Herstellung von Peptiden und Acylpeptiden, spektralphotometrische, chromatografische und MALDI-ToF/MS Analysen der Produkte sowie Charakterisierung der tensidischen Eigenschaften”; 2012, [“Chemoenzymatic preparation of peptides and acylpeptides, spectrophotometric, chromatographic and MALDI-ToF/MS analysis of the products and characterization of the surface-active properties”]) describes the enzymatic synthesis of di- and oligopeptides and the subsequent synthesis of acylated oligopeptides and the use thereof as surfactants.

Furthermore, enzyme-catalyzed polymerization processes have already been described in the field of the preparation of oligomers, which are considered for the production of bioplastics. A review article by Gübitz and Paulo (“New substrates for reliable enzymes: enzymatic modification of polymer”; Current Opinion in Biotechnology, 2003, 14: 577-582) mentions various approaches for the enzyme-catalyzed synthesis of natural and synthetic polymers.

In the dissertation by J. Duwensee (“Lipasen-katalysierte Polykondensation in wasserhaltigen Reaktionssystemen”; 2008, [“Lipase-catalyzed polycondensation in aqueous reaction systems”]) in particular a method for the preparation of polyesters by lipase-catalyzed polymerization reaction for the use e.g. as packaging material or in medical engineering is described.

The publications of Hilterhaus et al. (“Reactor Concept for Lipase-Catalyzed Solvent-Free Conversion of Highly Viscous Reactants Forming Two-Phase Systems”; Organic Process Research & Development, 2008, 12, 618-625) und Korupp et al. (“Scaleup of Lipase-Catalyzed . Polyester Synthesis”; Organic Process Research & Development, 2010, 14, 1118-1124) describe processes for producing polyester by lipase-catalyzed polymerization reaction.

DE 10 2005 026 135 A1 describes a method for preparing an aqueous polymer dispersion by enzyme-catalyzed reaction of a hydroxycarboxylic acid compound to a polyester in the presence of a dispersant from the group of emulsifiers and protective colloids.

All these known methods have in common, that the polymerization reaction for the preparation of oligomers and polymers obtainable therefrom is so far carried out in anhydrous or non-polar environment. This is achieved, for example, by using a water-free reactor system, azeotropic distillation of the occurring water from the reaction medium or by carrying out the reaction in non-polar organic solvents or by using non-polar solvent components and accordingly carrying out the reaction in an at least two-phase (binary) solvent system, comprising a polar aqueous phase and a non-polar organic phase.

In the methods described by Gübitz and Paulo, for example for the synthesis of phenolic polymers and acrylic polymers with oxidoreductases, a micelle solution is used. In the laccase-catalyzed synthesis of polyacrylamide and poly sodium acrylate, the addition of surfactants to form an emulsion is mentioned. Both, when using a micelle solution and in the case of the preparation of an emulsion, however, the reaction medium comprises a two-phase system consisting of an aqueous polar phase and a nonpolar phase. Further methods mentioned therein relating to further synthetic polymers merely affect their surface modifications (e.g. of polyester, polyamide or polyacrylonitrile).

The methods described in the dissertation of Duwensee (2008) exclusively make use of binary solvent systems consisting of an organic non-polar extraction phase and an aqueous (polar) reaction phase.

In the methods described by Hilterhaus et al. and Korupp et al. the synthesis is carried out in an anhydrous reactor system.

As already mentioned above, this is disadvantageous on the one hand from the aspect of procedural economy as well as on the other hand from the aspect of ecological process management.

In particular, if the known and above-described polymerization processes shall be carried out using bioengineered monomers or oligomers as starting materials, the problem arises that these bioengineered starting materials are typically present in aqueous reaction media and then for use in the known polymerization processes must be transferred in high purity into an anhydrous medium. In contrast, in the process of the present invention a high purity of the resulting materials is not absolutely necessary due to the high selectivity of the reaction system. In addition, a precise adjustment of the mixing ratio of the monomers, which is of crucial importance in the chemical reaction, is no longer necessary in the enzymatic polymerization reaction according to the present invention.

The new enzyme-catalyzed polymerization process according to the present invention is carried out in a single phase (polar) aqueous solution, which is particularly advantageous compared to the known methods because now for the first time the possibility exists to work with reaction media that are free from non-polar solvents. Furthermore, the new polymerization method is also particularly suitable when bioengineered monomers or oligomers are to be used as starting materials, as it is then possible to use the aqueous monomer/oligomer-containing fermentation supernatants after cell separation directly in the polymerization reaction for producing the prepolymers, without need for further purification prior to use. This allows a significant reduction of the process steps and thus improved process efficiency and economy can be achieved.

In particular, so far no methods for enzyme-catalyzed production of bio-based prepolymers having polyamide bonding structure or their use for the production of so-called bioplastics based on bio-based polyamide have been described.

### OBJECT TO BE SOLVED

The object of the present invention was to provide a new process for preparing prepolymers for the production of plastics, which avoids the disadvantages of methods known from the prior art. In addition, the new method should be characterized by improved procedural economy. In a further aspect of the invention, the novel process should be suitable to provide a process with high sustainability and it should be suitable for the production of bioplastics from completely and/or partially biobased mono- or oligomers based on renewable resources. In particular, the new process should allow the production of prepolymers with polyamide bonding structure for the production of new plastics based bio-based polyamide.

### DESCRIPTION OF THE INVENTION

The present invention relates to a process for preparing prepolymers for the production of plastics, wherein one or more different monomeric and/or oligomeric compounds are subjected to a polymerization reaction, which is characterized in that the polymerization reaction is carried out in a single phase (polar) aqueous solution with addition of one or more enzymes for catalyzing the polymerization reaction.

Therein, the monomeric and/or oligomeric compounds, being present in the aqueous reaction medium in dissolved form, are reacted as starting materials by enzyme-catalyzed reaction to longer polymers until a chain length is reached at which the formed polymers precipitate as a so-called prepolymer from the aqueous (polar) reaction solution.

Therein, a “prepolymer” according to the present invention—in contrast to an oligomer, which can be used as a possible starting material for preparing the prepolymers—indicates the molecule (polymer) formed from the monomers/oligomers in the polymerization reaction having such a chain length at which the formed molecule (polymer)—in contrast to the oligomer—precipitates from the aqueous reaction solution as the so-called prepolymer and may therewith be separated from the aqueous reaction mixture to be reacted later in subsequent reaction steps to longer linear or branched homopolymers or copolymers, polymer blends (plastics). The specific chain length, at which the formed polymer precipitates as a prepolymer from the aqueous monomer/oligomer-containing reaction solution on the one hand depends on the type of raw materials used and the prepolymers obtainable therefrom, and on the other hand on the specific reaction conditions such as temperature, pH-value or composition of the reaction medium.

The prepolymers according to the invention may generally be homopolymers or copolymers. The term homo-/copolymer is generally known to those skilled in the art.

Particularly preferred prepolymers of the present invention are those having a polyamide (polyamide type) bonding structure. Therein a polyamide or polyamide type bonding structure represents a bonding structure via a structural element of the general formula

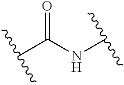

Also possible, but less preferred, are prepolymers having a polyester-type bonding structure. Therein a polyester-type bonding structure represents a bonding structure via a structural element of the general formula

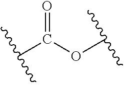

The term “plastic” refers in the conventional sense a polymeric solid article which is formed synthetically or semi-synthetically from the prepolymers formed according to the present invention. Therein, the obtainable plastics can consist of both linear and of branched and crosslinked chains.

Generally, a distinction is made between the three major groups of thermoplastics, thermosets and elastomers in plastics. According to the invention thermoplastic and thermosetting plastics are preferred, with thermoplastics being particularly preferred.

The method of the present inventive is in principle suitable for the preparation of prepolymers, such as polyesters (PES) comprising e.g. polybutylene terephthalate (PBT), polyethylene terephthalate (PET), polytrimethylene terephthalate (PTT), polyethylene naphthalate (PEN), polycarbonate (PC), and unsaturated polyester resin (UP), etc.; polyamides (PA) comprising e.g. polycaprolactam (Perlon, polyamide-6), nylon (polyamide 6.6; polyhexamethyleneadipic acid amide), PA 69 (hexamethylene diamine/azelaic acid), PA 612 (hexamethylenediamine/dodecanedioic acid), PA 11 (11 aminoundecanoic acid), PA 12 (laurolactam or w-amino dodecanoic acid), PA 46 (tetramethylenediamine/adipic acid), PA 1212 (dodecane diamine/dodecanedioic acid), PA 6/12 (caprolactam/laurolactam), PA 1010, etc.; polyethylene (PE) comprising high density polyethylene (PE-ND; HDPE), low density polyethylene (PE-LD; LDPE), linear low density polyethylene (PE-LLD; LLDPE), high molecular weight polyethylene (PE-HMW); ultrahigh molecular weight HDPE (PE-(UHMW)), etc.; as well as polypropylene (PP), polystyrene (PS), acrylonitrile-butadiene-styrene (ABS), styrene-acrylonitrile (SAN), polyoxymethylene (POM), polymethacrylate (PMA), polymethyl methacrylate (PMMA), polyvinyl chloride (PVC), polyphenylene ether (PPE), polyether ether ketone (PEEK), etc..

Preferred are prepolymers which are suitable for producing thermoplastics, such as in particular polyester (PES), polyamides (PA), acrylonitrile-butadiene-styrene (ABS), polymethacrylate (PMA), polymethyl methacrylate (PMMA), polycarbonate (PC), polyethylene terephthalate (PET), polyethylene (PE), polypropylene (PP), polystyrene (PS), polyetheretherketone (PEEK) and polyvinyl chloride (PVC), polyphenylene ether (PPE).

Very particularly preferred are prepolymers from the group of polyamides (PA) and polyester (PES), with polyamides being most preferred.

In the process of the present invention the prepolymers of the present invention are formed by catalytic polymerization of appropriate monomer and/or oligomer compounds. Therein, the same or different monomer and/or oligomer compounds having the same and/or different chain lengths may be reacted with each other. That means, for example, a monomer or oligomer compound can be used with unitary chain length or with components of different chain lengths. It is also possible to use two or more different monomer and/or oligomer compounds to react with each other, wherein one monomer or oligomer compound may comprise uniform chain lengths or components of different chain lengths and wherein the further monomer and/or oligomer compound(s) may also comprise uniform chain lengths or components of different chain lengths. It is also possible to use one or more monomer compounds, or one or more oligomer compounds or oligomer compounds and monomer compounds, respectively, to react with each other.

Monomers or monomer compounds usually refer to low molecular weight reactive molecules, which may combine to form linear or branched prepolymers or polymers. Monomers may be single substances, but also mixtures of different compounds, which in the first case form homopolymers and in the second case copolymers. Oligomers or oligomer compounds usually refer to molecules, which are built from a plurality of structurally identical or similar units (monomers), but which—compared to a prepolymer according to the present invention—are still soluble in water or the single-phase aqueous reaction medium and are thus still available as a reactant for an enzymatic reaction in the aqueous reaction solution.

In accordance with the present invention, preferred monomer and oligomer compounds are selected from the group comprising diamines, carboxylic acids, in particular hydroxy carboxylic acids, di- and tricarboxylic acids, fatty acids with low, medium and high chain length, amino carboxylic acids, caprolactams, particularly aminocaprolactams, glucose, lactones, polyols, diols, glycols, polyethylene glycols, glycerol, (di-, tri-, polyglycerol), mono-, di-, tricarboxylic acid esters, etc., and respective derivatives thereof, in particular ester derivatives thereof, such as particularly amino acid ester derivatives, and mixtures thereof. Particularly preferred monomer and oligomer compounds of the present invention are diamines, dicarboxylic acids, amino carboxylic acids, caprolactam, in particular aminocaprolactam, and carboxylic acids, especially citric acid, adipic acid, sebacic acid and succinic acid.

Examples of diamine compounds are linear or branched diaminoalkanes (H₂N—(C)ₙ—NH₂; with n≧4), in particular C₄-C₂₈ diaminoalkane, in particular C₄-C₂₀ diaminoalkanes, especially C₄-C₁₂ diaminoalkane, especially C₄-C₁₀, diaminoalkanes such as diaminobutanes, diaminopentanes, diaminoheptanes, diaminoohexanes, diaminoheptanes, diaminoctanes, diaminononanes, diaminodecanes, diaminoundecanes, diaminododecanes etc.; such as 1, 4-diaminobutane, 1,5-diaminopentane, 1,6-diaminohexane, 1,7-diaminoheptane, 1,8-diaminooctane, 1,9-diaminononane, 1,10-diaminodecane, 1,11-diaminoundecane, 1,12-diaminododecane, etc. Comprised are further the corresponding constitutional isomers of said diaminoalkanes and those, optionally being substituted with other substituents e.g. hydroxy. Particularly preferably the diamino compounds are selected from the group consisting of 1,4-diaminobutane and 1,5-diaminopentane.

Examples of dicarboxylic acids are C₂-C₂₈ alkane dicarboxylic acids, in particular C₂-C₁₆ alkane dicarboxylic acids and C₄-C₂₈ alkane dicarboxylic acids such as oxalic acid (ethanedioic acid), malonic acid (propanedioic acid) succinic acid (butanedioic acid), glutaric acid (pentanedioic acid), adipic acid (hexanedioic acid), pimelic acid (heptanedioic acid), suberic acid (octanedioic acid), azelaic acid (nonanedioic acid), sebacic acid (decanedioic acid), undecanedioic acid, dodecanedioic acid (decane-1,1 0-dicarboxylic acid), brassylic acid (tridecanedioic acid) tetradecanedioic acid, thapsic acid (hexadecanedioic acid) etc., as well as their corresponding constitutional isomers; C₃-C₂₈ alkene dicarboxylic acids, in particular C₃-C₁₆ alkene dicarboxylic acids, and their corresponding constitutional isomers as well as those of the aforementioned groups, which may be substituted with one or more, especially one or two hydroxy, keto or amino groups such as tartronic acid, tartaric acid, malic acid, a-ketoglutaric acid, oxaloacetic acid, phthalic acid, isophthalic acid, terephthalic acid, glutamic acid, aspartic acid, maleic acid, fumaric acid, and diphenyl ether-4,4-dicarboxylic acid, naphthalene-1,4-dicarboxylic acid, naphthalene-2,6-dicarboxylic acid, and hexahydroterephthalic acid. Comprised are further the corresponding constitutional isomers of the carboxylic acids as well as those which may optionally be substituted with further substituents. Particularly preferably the dicarboxylic acids are selected from the group consisting of 1,6-hexanedioic acid and 1,1 0-decanedioic acid.

Examples of tricarboxylic acids are e.g. citric acid, isocitric acid, aconitic acid (1,2,3-propenetricarboxylic acid) carballylic acid (1,2,3-propanetricarboxylic acid), benzotricarboxylic acids such as trimesic acid, hemimellitic acid and trimellitic acid.

Examples of hydroxy carboxylic acids include carboxylic acids containing at least one carboxy group as well as one or more hydroxy group(s) such as α-, β- and γ-hydroxy carboxylic acids. Examples of hydroxy carboxylic acids are, in addition to the above mentioned hydroxy di- tricarboxylic acids e.g. glycolic acid, mandelic acid, lactic acid, hydroxybutyric acid, polyhydroxy butyric acid, mevalonic acid, gallic acid, 4-hydroxybutanoic acid, 2-hydroxybenzoic acid (salicylic acid), 4-hydroxybenzoic acid. Comprised are further those of the above-mentioned compounds, which may optionally be substituted with further substituents.

Examples of amino carboxylic acids include carboxylic acids containing at least one carboxy group as well as one or more amino group(s). Examples are C₁-C₂₀ amino carboxylic acids, especially C₂-C₂₀ amino carboxylic acids, preferably C₅-C₂₀ amino carboxylic acids such as α-, β- and γ-amino acids such as the essential amino acids alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine; and amino carboxylic acids deriving from an amino-substituted mono-, di- or tricarboxylic acid, in particular as defined above, such as for example from the above-mentioned di- or tricarboxylic acids which are substituted with one or more amino groups such as amino adipic acid; and respective derivatives thereof, in particular amino acid ester derivatives thereof. Further comprised are those of the abovementioned compounds, which may optionally be substituted with further substituents.

Examples of dicarboxylic acid esters include esters of the above mentioned dicarboxylic acids which formally are composed of a dicarboxylic acid, as defined above, and an alcohol or phenol. Also comprised are the corresponding constitutional isomers of said dicarboxylic acid ester as well as those which may optionally be substituted with further substituents.

Examples of diols include C₂-C₂₈ alkanediols, especially C₂-C₁₆ alkanediols such as 1,2-, 1,3-, 1,4-alkanediols, etc., for example the corresponding ethane, propane, butanediols such as 1,2-ethanediol (ethylene glycol), 1,2-propanediol (propylene glycol), 1,3-propanediol (1,3-propylene glycol), 1,2-butanediol, 1,3-butanediol, 1,4-butanediol, 2,3-butanediol, neopentyl glycol, etc. as well as their corresponding constitutional isomers, as well as a, w-diols occurring by condensation of ethylene glycol such as diethylene glycol, triethylene glycol, polyethylene glycol etc., as well as for example diethylene glycol, cyclohexanedimethanol, 2,2-bis(4-hydroxyphenyl) propane and 2,2-bis(4-hydroxyethoxyphenyl) propane. Comprised are further those of the above-mentioned compounds, which may optionally be substituted with further substituents.

In the sense of the present invention the above definitions comprise the corresponding possible stereoisomeric configurations (enantiomers, diastereomers as well as their racemates; α-, β-, γ-, D-, L-configurations). According to the invention also derivatives of the above mentioned compound are comprised, which due to their specific functional groups are suitable to be used in accordance with the method of the present invention. Further, the invention comprises also those of the above-mentioned compounds, which may optionally be substituted with further substituents, provided that the functionality of the relevant reactive groups is not impaired.

Particularly preferably, the monomer and oligomer compounds are selected from the group of diamines, dicarboxylic acids, amino carboxylic acids and their ester derivatives, hydroxycarboxylic acids, caprolactams and/or dicarboxylic acid esters.

In another particularly preferred embodiment, the monomer compound is selected from one or more diamine compounds from the group of diamino alkanes, especially C₄-C₁₀-diaminoalkanes, preferably C₄-C₆-diaminoalkanes, one or more dicarboxylic acids, especially C₆-C₂₈-dicarboxylic acids, preferably C₆-C₁₀-dicarboxylic acids, one or more tricarboxylic acids, one or more amino carboxylic acids, especially C₂-C amino carboxylic acids, preferably C₅-C₂₀-amino carboxylic acids, one or more hydroxy carboxylic acids and/or one or more caprolactams, especially aminocaprolactams, each as defined above.

In a further particularly preferred embodiment, the monomer compound selected from the group of diaminoalkanes is 1,5-diaminopentane, from the group of carboxylic acids is citric acid, adipic acid, sebacic acid or succinic acid, from the group of amino carboxylic acids is aminoadipic acid and ester derivatives thereof, in each case as defined above, and/or mixtures thereof.

Very particularly preferred are those monomer and oligomer compounds, which are suitable for the preparation of prepolymers with polyamide bonding structure.

For the particularly preferred preparation of prepolymers with polyamide bonding structure it is further preferred to select the monomer and/or oligomer compound from the group of diamines, dicarboxylic acids, amino carboxylic acids and ester derivatives thereof, caprolactam and aminocaprolactam. Therein, it is on the one hand preferred to select a mixture of dicarboxylic acids and diamines (which may comprise the same or different compounds, each with the same or different chain length), wherein the two relevant groups of the polyamide bonding structure, namely, the carbonyl and the amide group, are formed from two different components, namely on the one hand from the dicarboxylic acid and on the other hand from the diamine. It is also preferred to select the monomer and/or oligomer compound from the group of amino carboxylic acids, as defined above, (wherein one or a mixture of several amino carboxylic acids, each with the same or different chain lengths are comprised) wherein both relevant groups for forming the polyamide bonding structure are virtually present in a single building block.

Regarding the particularly preferred diamines, carboxylic acids (in particular dicarboxylic acids) and amino carboxylic acids reference can be made to the definitions above.

In the process for the preparation of prepolymers with polyester-type bonding structure, which is also possible according to the present invention, the monomer and/or oligomer compounds are preferably selected from the groups of the di- and tricarboxylic acids and derivatives thereof, of dialcohols (diols) and glycerol, all as defined above. Preferred is a mixture comprising one or more carboxylic acid compounds, such as in particular citric acid, adipic acid, sebacic acid or succinic acid and one or more diols such as, in particular, 1,4-butanediol and/or glycerol. Regarding the particularly preferred di- and tricarboxylic acids and dialcohols (diols) reference can be made to the definitions above.

The process of the present invention is characterized by an enzyme-catalyzed polymerization. Therein, in principle all enzymes can be used which are suitable to catalyze the reaction of the selected monomer and/or oligomer compounds to the desired prepolymer, in particular those from the group of hydrolases (enzyme class EC3), the oxidoreductases (enzyme class EC1) and the lyases (enzyme class EC4). Particularly preferred are enzymes from the group of hydrolases (enzyme class EC3).

Examples of suitable enzymes from the group of hydrolases include e.g. peptidases, (also proteases, proteinases), nucleases, phosphatases, glycosidases, esterases, lipases, lactamases, amidases, (amino)caprolactamases, polyamidases, carboxylesterase, carboxypeptidases, amylases, etc., and mixtures thereof.

Examples of suitable enzymes from the group of oxidoreductases include e.g. oxidases, dehydrogenases and reductases, such as alcohol dehydrogenase, glucose oxidase, aldehyde dehydrogenase, dihydrofulate reductase, nitrite reductase, ferredoxin-nitrite reductase, sulfite oxidase, polyphenol oxidase, catalase, xanthine oxidase etc. and mixtures thereof.

Preferably, from the group of lyases decarboxylase, more preferably L- or D-amino adipic acid decarboxylase, is selected.

Preferred enzymes from the group of hydrolases are e.g. peptidases, phosphatases, glycosidases, esterases, lipases, lactamases, amidases, (amino)caprolactarnases, polyamidases, carboxyl esterase, and mixtures thereof.

Very particularly preferred are enzymes which are suitable for the preparation of prepolymers with polyamide bonding structure.

In the particularly preferred process of the present invention for preparing prepolymers with polyamide bonding structure, the enzymes are preferably selected from the group of amidases, polyamidases, lactamases and (amino)caprolactamases. Very particularly preferred the enzyme is the protease “subtilisin A” from Bacillus licheniformis.

In the process for the preparation of prepolymers with polyester-type bonding structure, which is also possible according to the present invention, the enzymes are preferably selected from the group of peptidases and proteases.

The process of the present invention is especially characterized in that the polymerization reaction is carried out in a single phase (polar) aqueous solution. In the sense of the present invention an aqueous solution is a hydrophilic water-based reaction solution, which is essentially free from non-polar organic solvents or extracting agents. Therein, the aqueous reaction solution of the present invention is in particular also free of other non-polar (lipophilic) solvent components. Therein, the term “non-polar organic solvent or extracting agent” refers to those solvents which are known to be immiscible with water. The term “other non-polar solvent components” refers to those solvent additives which are suitable to form a non-polar phase in the aqueous (polar) reaction medium, such as in particular micelle forming or vesicle, liposome and emulsion-forming substances. Accordingly, the aqueous solution may, for example, also be free of micelle, vesicle or liposome-forming substances and/or may be free of such micelles, vesicles and liposomes, as well as of emulsions.

Carrying out a process in single-phase aqueous reaction systems is so far not possible with the known methods, since the reaction of the monomers with each other usually occurs together with elimination of water, and accordingly an appropriate shift of the reaction equilibrium to the side of the polymers by dehydration is carried out.

In methods of enzyme-catalyzed polymerization, which by virtue of the solubility and activity of the enzymes in water mandatorily must be carried out in an aqueous medium, the polymerization with elimination of water can nevertheless be achieved as the dissolved monomeric or oligomeric starting materials sort of diffuse into the protein molecule (enzyme) and suffer from such a charge transfer at its active site, so that the polymerization with elimination of water, which is necessary and desired in the polymerization reaction, can occur, quasi in the space shielded by the aqueous reaction medium or under protection by the protein molecule.

When working in purely aqueous systems, however, the effect occurs that the polymers formed in the polymerization reaction are insoluble in water, even at a relatively low chain length, and precipitate in this purely aqueous reaction medium, thus not being available any longer for further chain extensions by polymerization. In the previously known methods such precipitation of the prepolymers is not desired for the above reasons and is avoided by using an at least two-phase solvent system, wherein the prepolymers being insoluble in the polar aqueous phase remain dissolved in the non-polar phase. In contrast, however, the process of the present invention explicitly aims at precipitating the formed polymers as so-called prepolymers from the aqueous reaction medium, then either separate them therefrom or directly use them in this medium, as described above and further below.

The aqueous solution, which is subjected to polycondensation reaction according to the invention, consists essentially of water, although amounts of polar solvents may be added. The addition of polar solvents is then selected such that the monomers and/or oligomers are present at the active site of the enzyme, relative to their charge in an activated condition for the polymerization reaction (in the sense of an appropriate shift of charge), without impairing the enzyme activity.

Added polar solvents according to the invention include, for example, methanol and ethanol. Ethanol is preferably added.

It is also possible to add surface-active substances in the sense of a shift of charge at the active site of the enzyme, for example, emulsifiers or surfactants. Also the addition of pH-regulating substances, buffers or the variation of the salt or ion concentration is possible. By appropriate addition of such substances, it is possible to positively influence the thermodynamic equilibrium of the enzyme-catalyzed polymerization reaction from hydrolysis towards the synthesis of the prepolymers.

A single-phase aqueous solvent system according to the present invention refers to an exclusively polar solvent system, which is completely and homogeneously miscible with water. Preferably, such a single-phase aqueous solvent system does not comprise any non-polar phases or areas.

It is also possible to influence the thermodynamic reaction equilibrium and thus appropriately shift the process towards the desired direction of the synthesis of the prepolymers by the selection of the technical process parameters such as appropriate temperature and pressure settings, the selection of appropriate reaction times and via the amounts of enzyme and/or monomers/oligomers (surplus/deficit) used. Either only single of the aforementioned parameters or several of the parameters in any combination with each other can be varied and adjusted appropriately. The polymerization reaction is in principle a known reaction method, which is illustrated below by way of an example of a general reaction scheme for the preferred methods for the preparation of prepolymers having polyamide-type and polyester-type bonding structures according to the present invention. Therein, in each case,
R is hydrogen and/or a suitable substituent, which in the case of n>1 may be the same or different at the different positions,
n is an integer ≧1,
m is an integer ≧1.

Enzyme-catalyzed synthesis of prepolymers with polyimide-type bonding structure from a mixture of a diamine and a dicarboxylic acid:

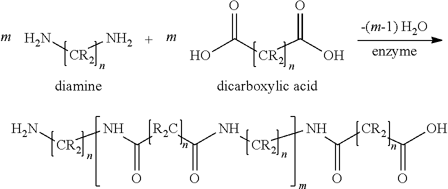

Enzyme-catalyzed synthesis of prepolymers with polyamide-type bonding structure from an amino carboxylic acid compound:

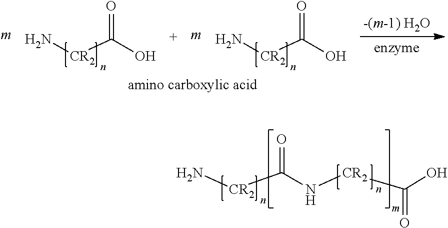

Enzyme-catalyzed synthesis of prepolymers with polyester-type bonding structure from a dicarboxylic acid and a diol compound:

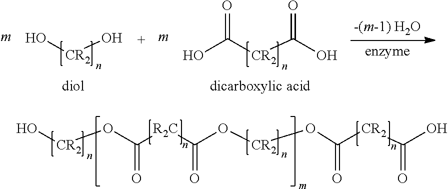

Enzyme-catalyzed synthesis of prepolymers with polyester-type bonding structure from a hydroxycarboxylic acid compound:

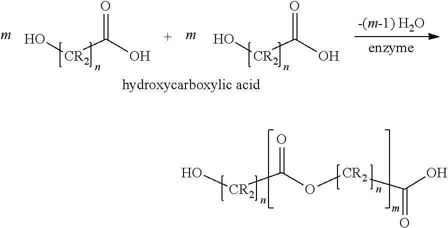

The above illustrations of the reaction pathways represent merely an exemplary illustration of the basic reaction principles and act in no way limiting.

The prepolymers formed in the polymerization process according to the present invention are usually precipitated from the aqueous reaction solution, separated from the aqueous supernatant by known methods such as centrifugation or filtration and optionally processed in subsequent steps to thermoplastics or thermosetting plastics and optionally further processed by known processing methods to obtain plastic articles, for example in spinning processes or in thermoplastic molding processes, in particular in injection molding, casting or extrusion processes.

Thus, the process of the present invention preferably comprises the steps of:
a) preparing one or more monomer or oligomer compounds, for example by fermentation, enzymatic reaction or chemical synthesis, wherein fermentation and enzymatic reaction are preferred,
b) separating the aqueous supernatants with the dissolved monomer or oligomer compounds,
c) adding one or more enzymes, which catalyze the polymerization reaction of the monomer or oligomer compounds, to the aqueous solution containing the monomer or oligomer compounds,
d) precipitation of the prepolymers from the aqueous reaction solution,
e) separating the precipitated prepolymers, preferably by centrifugation or filtration,
f) optionally further processing of the prepolymers to plastics, and
g) optionally further processing of the resulting plastics into plastic articles, preferably in spinning processes or thermoplastic molding processes, in particular in injection molding, casting or extrusion processes.

Alternatively, monomer and/or oligomer compounds from commercially available sources such as petrochemically produced monomer/oligomer compounds can be used, which are then dissolved in water and directly supplied in step c).

In a particularly preferred process according to the present invention, the monomer or oligomer compounds used are obtained by means of biotechnological processes, in particular by fermentation or enzymatic reaction. This is particularly advantageous from the viewpoint of sustainable process management, since thereby the commonly used petrochemical raw materials can be replaced by sustainable biotechnologically produced raw materials. As with the process according to the present invention for the first time a process management in aqueous reaction medium is possible, the use of starting materials being prepared by fermentation or enzymatically is particularly suitable. Usually in the fermentative or enzymatic production of the monomer and oligomer compounds used in the present invention those occur dissolved in the aqueous fermentation supernatant or in the aqueous reaction medium. These can, if necessary after removal of the cells, be used directly, usually without the need for further reprocessing, with the monomer or oligomer compounds dissolved therein for further processing, by directly initiating the polymerization reaction in this aqueous fermentation supernatant or reaction medium by the addition of the enzymes. At a certain chain length the corresponding prepolymers precipitate from said aqueous reaction solution and can be separated and used for further processing as described above. Very preferably, monomer or oligomer compounds are used which are obtained by fermentation and these are then preferably supplied accordingly directly in the aqueous fermentation supernatant to the polycondensation reaction.

Accordingly, the process according to the present invention is particularly suitable for the preparation of prepolymers from monomer or oligomer compounds prepared by biotechnological methods, in particular by fermentation. In a particular embodiment thereof prepolymers with polyamide-type bonding structure are prepared, wherein as the monomer compound e.g. diaminopentane is used as the diamine compound, which is obtainable by fermentation using a recombinant microorganism, such as a recombinant bacterium belonging to the species Corynebacterium glutamicum.

Therein, due to the possible use of biotechnologically produced raw materials with high sustainability, as a substitute for fossil fuels, the prepolymers obtainable therefrom are in the purposes of the present invention also referred to as bio-based prepolymers, and the thermoplastics and thermosetting plastics made from these bio-based prepolymers are referred to as bio-based plastics or bioplastics.

A further aspect of the present invention relates to the use of the prepolymers obtainable by the process of the present invention for the production of plastics, in particular thermoplastics or thermosetting plastics, and plastic articles obtainable therefrom. Therein, a particularly preferred embodiment is directed to the use of the prepolymers obtainable by the process of the present invention for the production of plastics (bioplastics).

Another aspect of the present invention relates to the use of the prepolymers obtainable by the process of the present invention for the production of plastics and plastic articles, wherein the plastic articles are obtained by spinning processes, thermoplastic or thermosetting molding processes, especially in injection molding, casting or extrusion processes. Again, a particularly preferred embodiment of the invention relates to the respective bio-based prepolymers and bioplastics.

A further aspect of the present invention relates to the aforementioned use of the invention for the production of textiles, thermoplastic molded articles, packaging materials and building materials, all in particular by using the bioplastics of the present invention.

By the particularly preferred preparation of prepolymers with polyamide-type bonding structure accordingly, the plastics (bioplastics) and plastic articles obtainable therefrom are inevitably polyamide-based plastics (bioplastics) or plastic products, which are preferred in the present invention accordingly.

The present invention in particular encompasses the following embodiments:
1. A process for preparing prepolymers for the production of plastics, wherein one or more monomer or oligomer compounds are subjected to a polycondensation reaction, which is characterized in that the polycondensation reaction is carried out in a single phase aqueous solution with the addition of one or more enzymes catalyzing the polymerization reaction.
2. The process of embodiment 1, wherein the prepolymers are precipitated from the single-phase aqueous reaction solution and then separated therefrom and further processed into plastics.
3. The process according to one of the preceding embodiments for the preparation of prepolymers for the production of plastics which are selected from the group of thermoplastics and thermosetting plastics.
4. The process according to one of the preceding embodiments, wherein the prepolymer has a polyamide-type or a polyester-type bonding structure, preferably the prepolymer is polyamide (PA) or polyester.

5. The process according to one of the preceding embodiments wherein the monomer and/or oligomer compounds are selected from the group comprising diamines, carboxylic acids, in particular hydroxycarboxylic acids, di- and tricarboxylic acids, amino carboxylic acids, caprolactams, particularly aminocaprolactams, lactones, diols, glycerol and derivatives and mixtures thereof, respectively.

6. The process according to one of the preceding embodiments, wherein the enzymes are selected from the group of hydrolases, oxidoreductases and lyases, preferably from the group of hydrolases.

7. The process according to one of the preceding embodiments, wherein
a) (i) the prepolymer has a polyamide-type bonding structure and
   (ii) as monomers or oligomers a mixture of one or more diamine compounds with one or more dicarboxylic acid compounds, one or more amino carboxylic acids or esters thereof, or caprolactam, in particular aminocaprolactam, and
   (iii) as enzyme a hydrolase, preferably a polyamidase or (amino)caprolactamase is used, or wherein
b) (i) the prepolymer has a polyester-type bonding structure and
   (ii) as monomers or oligomers a mixture of one or more diols, particularly 1,4 butanediol and/or glycerol with one or more carboxylic acid compounds, in particular citric acid, adipic acid and/or sebacic acid, succinic acid, and
   (iii) as enzyme a hydrolase, preferably a protease or peptidase is used.

8. The process according to one of the preceding embodiments, wherein the monomer or oligomer compounds are prepared by fermentation, preferably by fermentation using a recombinant microorganism.

9. The process according to one of the preceding embodiments, comprising the steps
a) preparing one or more monomer or oligomer compounds, preferably by fermentation or enzymatic reaction,
b) separating the aqueous supernatants with the monomer or oligomer compounds dissolved therein,
c) adding one or more enzymes catalyzing the polymerization reaction of the monomer or oligomer compounds to the aqueous solution containing one or more monomer or oligomer compounds,
d) precipitating the prepolymers from the aqueous reaction solution,
e) separating the precipitated prepolymers, preferably by centrifugation or filtration,
f) optionally further processing of the separated prepolymers to plastics, and
g) optionally further processing of the resulting plastics into plastic articles, preferably in spinning processes or thermoplastic or thermosetting molding processes, in particular in injection molding, casting or extrusion processes.

10. Use of the prepolymers obtainable by the process according to one of the preceding embodiments for the production of plastics, as well as plastic articles obtainable therefrom, in particular textiles, thermoplastic molded articles, packaging materials and building materials.

### EXAMPLES

In the following the invention is further illustrated by way of example. For the skilled person it is apparent that this example is exemplary only and will not narrow the scope of the invention.

For the synthesis of polymerization products based on dicarboxylic acids and diamines hydrolases from the EC-group 3 were used. The commercially available protease “subtilisin A” from Bacillus licheniformis A (company Megazyme; Order-No.: E-BSPRT) was used. The enzyme stock solution was 300 U/ml. The pH optimum of this enzyme lies at pH 7 -7.5, the pH stability lies at pH 5.5 -10.0 and the temperature optimum lies at 60° C.

Unless stated otherwise, all solutions used were applied in double-distilled water (ddH₂O). As dicarboxylic acid 2,6-hexanedioic acid and 1,10-decanedioic acid were used. A 1M solution of 1,6-hexanedioic acid was prepared at 60° C. 1,10-decanedioic acid was either dissolved as a 200 mM solution in 99.8% ethanol or prepared as a 2.5 mM solution in water. As diamines 1,4-diaminobutane and 1,5-diaminopentane were used. Of each a 400 mM solution in water was prepared.

The enzymatic synthesis was carried out under shaking in test tubes with screw cap at 60° C. and 1500 rpm for 50-60 h in a preheated thermal shaker (“Thermo Shaker Incubator” MS-100). Therefore 100 μl enzyme solution were added to 1000 μl of the 1,4-diaminobutane and 62.2 μl of the 200 mM decanedioic acid (in ethanol). In order to achieve the total volume of 2.1 ml, 969 μl ddH₂O was added.

Alternatively, 1000 μl 1,4-diaminobutane or 1,5-diaminopentane and 100 μl enzyme was were to 1000 μl of the 2.5 mM decanedioic acid solution and filled with ddH₂O. Similarly, Likewise, 500 μl 1,6-hexanedioic acid solution and 100 μl enzyme were added to 1000 μl 1,5-diaminopentane or 1,4-diaminobutane and filled with ddH₂O.

The initial pH value in the reaction was pH 5.5 in the case of hexanedioic acid and pH 10.0 in the case of decanedioic acid.

For the subsequent analysis, the synthesized samples were evaporated on a rotary evaporator under reduced pressure at 50 mbar at 60° C. From the evaporated synthesis batches 5-8 mg were weighed in each case and in about 1.5 ml HFIP solution (99.9% hexafluoroisopropanol, 0.1 wt % potassium trifluoroacetate) redissolved by stirring for several hours and then filtered (PTFE membrane 0.2 μm). The molecular weight (Mn and MW) and the dispersity of the synthesized products were determined by HFIP gel permeation chromatography (GPC HFIP). Depending on the synthesis batch both, prepolymers with smaller masses of about 700 -1300 Daltons as well as large polymers with masses between 100,000 and 300,000 Daltons were obtained; the dispersity was in each case below 1.25.

For further analysis, the evaporated synthesis batches were redissolved in about 200 μl HFIP solution and precipitated in an excess of cold methanol. Formed products precipitated; the supernatant containing the reactants was discarded. The samples were dried and re-examined using HFIP-GPC as well as IR spectroscopy, wherein in particular the short chain lengths of the prepolymers of 700-1300 Daltons were confirmed.

1. A process for preparing prepolymers for the production of plastics, wherein one or more monomer or oligomer compounds are subjected to a polycondensation reaction, which is characterized in that the prepolymer has a polyimide-type bonding structure and the polycondensation reaction is carried out in a single phase aqueous solution with the addition of one or more enzymes catalyzing the polymerization reaction.

2. The process of claim 1, wherein the prepolymers are precipitated from the single-phase aqueous reaction solution and then separated therefrom and further processed into plastics.

3. The process according to claim 1 for the preparation of prepolymers for the production of plastics which are selected from the group of thermoplastics and thermosetting plastics.

4. The process according to claim 1, wherein the prepolymer is polyimide.

5. The process according to claim 1, wherein the monomer and/or oligomer compounds are selected from the group comprising diamines, carboxylic acids, in particular hydroxycarboxylic acids, di- and tricarboxylic acids, amino carboxylic acids, caprolactams, particularly aminocaprolactams, and derivatives and mixtures thereof, respectively.

6. The process according to claim 1, wherein the enzymes are selected from the group of hydrolases, oxidoreductases and lyases, preferably from the group of hydrolases.

7. The process according to claim 1, wherein
(i) the prepolymer has a polyimide-type bonding structure and
(ii) as monomers or oligomers a mixture of one or more diamine compounds with one or more dicarboxylic acid compounds, one or more amino carboxylic acids or esters thereof, or caprolactam, in particular aminocaprolactam, and
(iii) as enzyme a hydrolase, preferably a polyamidase or (amino)caprolactamase is used.

8. The process according to claim 1, wherein the monomer or oligomer compounds are prepared by fermentation, preferably by fermentation using a recombinant microorganism.

9. The process according to claim 1, comprising the steps
a) preparing one or more monomer or oligomer compounds, preferably by fermentation or enzymatic reaction,
b) separating the aqueous supernatants with the monomer or oligomer compounds dissolved therein,
c) adding one or more enzymes catalyzing the polymerization reaction of the monomer or oligomer compounds to the aqueous solution containing one or more monomer or oligomer compounds,
d) precipitating the prepolymers from the aqueous reaction solution,
e) separating the precipitated prepolymers, preferably by centrifugation or filtration,
f) optionally further processing of the separated prepolymers to plastics, and
g) optionally further processing of the resulting plastics into plastic articles, preferably in spinning processes or thermoplastic or thermosetting molding processes, in particular in injection molding, casting or extrusion processes.

10. Use of the prepolymers obtainable by the process according claim 1 for the production of plastics, as well as plastic articles obtainable therefrom, in particular textiles, thermoplastic molded articles, packaging materials and building materials.

11. The process according to claim 2 for the preparation of prepolymers for the production of plastics which are selected from the group of thermoplastics and thermosetting plastics.

12. The process according to claim 2, wherein the prepolymer is polyamide.

13. The process according to claim 2, wherein the monomer and/or oligomer compounds are selected from the group comprising diamines, carboxylic acids, in particular hydroxycarboxylic acids, di- and tricarboxylic acids, amino carboxylic acids, caprolactams, particularly aminocaprolactams, and derivatives and mixtures thereof, respectively.

14. The process according to claim 2, wherein the enzymes are selected from the group of hydrolases, oxidoreductases and lyases, preferably from the group of hydrolases.

15. The process according to claim 2, wherein
(i) the prepolymer has a polyamide-type bonding structure and
(ii) as monomers or oligomers a mixture of one or more diamine compounds with one or more dicarboxylic acid compounds, one or more amino carboxylic acids or esters thereof, or caprolactam, in particular aminocaprolactam, and
(iii) as enzyme a hydrolase, preferably a polyamidase or (amino)caprolactamase is used.

16. The process according to claim 2, wherein the monomer or oligomer compounds are prepared by fermentation, preferably by fermentation using a recombinant microorganism.

17. The process according to claim 2, comprising the steps
a) preparing one or more monomer or oligomer compounds, preferably by fermentation or enzymatic reaction,
b) separating the aqueous supernatants with the monomer or oligomer compounds dissolved therein,
c) adding one or more enzymes catalyzing the polymerization reaction of the monomer or oligomer compounds to the aqueous solution containing one or more monomer or oligomer compounds,
d) precipitating the prepolymers from the aqueous reaction solution,
e) separating the precipitated prepolymers, preferably by centrifugation or filtration,
f) optionally further processing of the separated prepolymers to plastics, and
g) optionally further processing of the resulting plastics into plastic articles, preferably in spinning processes or thermoplastic or thermosetting molding processes, in particular in injection molding, casting or extrusion processes.

18. The process according to claim 3, wherein the enzymes are selected from the group of hydrolases, oxidoreductases and lyases, preferably from the group of hydrolases.

19. The process according to claim 3, wherein
(i) the prepolymer has a polyamide-type bonding structure and
(ii) as monomers or oligomers a mixture of one or more diamine compounds with one or more dicarboxylic acid compounds, one or more amino carboxylic acids or esters thereof, or caprolactam, in particular aminocaprolactam, and
(iii) as enzyme a hydrolase, preferably a polyamidase or (amino)caprolactamase is used.

20. The process according to claim 3, wherein the monomer or oligomer compounds are prepared by fermentation, preferably by fermentation using a recombinant microorganism.
